(19) United States
(12) Patent Application Publication
(10) Pub. No.: US 2009/0069452 A1
(43) Pub. Date: Mar. 12, 2009

(54) METHODS AND APPARATUS FOR PRODUCING ETHANOL FROM SYNGAS WITH HIGH CARBON EFFICIENCY

(75) Inventor: Heinz Juergen ROBOTA, Arvada, CO (US)

Correspondence Address: Range Fuels, Inc.;Attn: Ryan O'Connor, 11101 West 120th Ave. Suite 200, Broomfield, CO, 80021, US

(73) Assignee: Range Fuels, Inc, Broomfield, CO (US)

(21) Appl. No.: 12/198,208

(22) Filed: Aug. 26, 2008

Related U.S. Application Data
(60) Provisional application No. 60/970,660, filed on Sep. 7, 2007.

(51) Int. Cl. C07C 27/06; B01J 19/00

(52) U.S. Cl. 518/713; 518/728; 422/211

(57) ABSTRACT

The present invention discloses and teaches new methods of converting syngas into ethanol and/or other higher alcohols. Preferred embodiments recycle methanol, partially convert it to syngas, and then convert this additional syngas also to C₂₊ alcohols. Generally, the invention provides reactors comprising catalysts capable of converting syngas to alcohols with low selectivities to carbon dioxide and methane, and further provides process strategies to separate and recycle unreacted syngas as well as methanol produced by the catalyst. The invention is capable of turning modest per-pass reaction selectivities to a particular alcohol, such as ethanol, into economically significant net selectivities and yields.

### FIELD OF THE INVENTION

The present invention generally relates to the field of processes for the chemical conversion of synthesis gas to alcohols, such as ethanol.

### BACKGROUND OF THE INVENTION

Synthesis gas (hereinafter referred to as syngas) is a mixture of hydrogen (H₂) and carbon monoxide (CO). Syngas can be produced, in principle, from virtually any material containing carbon. Carbonaceous materials commonly include fossil resources such as natural gas, petroleum, coal, and lignite; and renewable resources such as lignocellulosic biomass and various carbon-rich waste materials. It is preferable to utilize a renewable resource to produce syngas because of the rising economic, environmental, and social costs associated with fossil resources.

There exists a variety of conversion technologies to turn these feedstocks into syngas. Conversion approaches can utilize a combination of one or more steps comprising gasification, pyrolysis, steam reforming, and/or partial oxidation of a carbon-containing feedstock.

Syngas is a platform intermediate in the chemical and biorefining industries and has a vast number of uses. Syngas can be converted into alkanes, olefins, oxygenates, and alcohols. These chemicals can be blended into, or used directly as, diesel fuel, gasoline, and other liquid fuels. Syngas can also be directly combusted to produce heat and power.

Since the 1920s it has been known that mixtures of methanol and other alcohols can be obtained by reacting syngas over certain catalysts (Forzatti et al., Cat. Rev.-Sci. and Eng. 33(1-2), 109-168, 1991). Fischer and Tropsch observed around the same time that hydrocarbon-synthesis catalysts produced linear alcohols as byproducts (Fischer and Tropsch, Brennst.-Chem. 7:97, 1926).

Today, almost half of all gasoline sold in the United States contains ethanol (American Coalition for Ethanol, www.ethanol.org, 2006). The ethanol in gasoline and other liquid fuels raises both the oxygen and the octane content of the fuels, allowing them to burn more efficiently and produce fewer toxic emissions.

In efforts to produce ethanol, or other alcohols, the overall process efficiency is affected by the selectivity with which a given carbon source can be converted to ethanol, rather than other carbon-containing molecules. It is desirable to selectively convert as much CO and H₂ into ethanol as possible, respecting thermodynamic limitations.

While a variety of existing catalyst systems can make ethanol from syngas, the associated efficiencies vary considerably. For example, U.S. Pat. No. 4,882,360 (Stevens) discloses that approximately 15% of the carbon converted from CO appears as ethanol. A large fraction of nearly half the carbon that is converted appears as carbon dioxide (CO₂) and methane (CH₄). Both CO₂ and CH₄ can theoretically be recycled and rerouted into ethanol through steam reforming, reverse water-gas shift, and other reactions. There is, however, considerable inefficiency and cost in separation, recompression, and endothermic chemistry to generate CO or H₂ from CH₄ and/or CO₂.

Recently, Hu et al. disclosed a modified methanol-synthesis catalyst comprising copper (Cu), zinc (Zn), aluminum (Al), and cesium (Cs), which was compared with rhodium-based alcohol-synthesis catalysts. Results were obtained with a granular 70-100 mesh Cu—Zn—Al—Cs catalyst at 280° C., 53 atm, H₂/CO=2, and a space velocity of 3750 hr¹. These results indicated 30% selectivity to ethanol, 57% selectivity to methanol, and 13% selectivity to other hydrocarbons and oxygenates, with less than 1% combined selectivity to CH₄ and CO₂, at about 35% CO conversion (Hu et al., Catalysis Today 120, 90-95, 2007; incorporated herein by reference).

To address the deficiency in the art, improved methods, and apparatus for carrying out those methods, are needed for selectively producing ethanol and other C₂₋ alcohols from syngas. Improved methods and apparatus should effectively deal with methanol, when methanol is not a desired product. Methanol formation is significant under most (if not all) relevant process conditions for turning syngas into C₂₊ alcohols such as ethanol.

What is especially needed is an invention that discloses and teaches a new and non-obvious manner of converting syngas into ethanol, in good selectivities, wherein most of the methanol can ultimately also be channeled to ethanol.

### SUMMARY OF THE INVENTION

In one aspect of the present invention, methods are provided for producing at least one C₂-C₄ alcohol from syngas, the methods comprising:

(i) providing a reactor comprising a catalyst capable of converting syngas to alcohols;

(ii) providing a first stream containing syngas having a H₂/CO ratio;

(iii) flowing the first stream into the reactor at reaction conditions effective for producing a second stream comprising methanol and the at least one C₂-C₄ alcohol, wherein the combined reaction selectivity to CO₂ and CH₄ is less than about 10%;

(iv) separating at least some unreacted syngas from the second stream;

(v) separating at least some methanol from the second stream;

(vi) recycling at least some of the unreacted syngas and some of the methanol back to the reactor; and

(vii) collecting a mixture comprising the at least one C₂-C₄ alcohol.

In some embodiments, the combined reaction selectivity to CO₂ and CH₄ is less than about 5%, preferably less than about 1%. The reaction selectivity to CO₂ individually is less than about 5%, preferably less than about 0.5%, and more preferably essentially 0, in certain embodiments. The reaction selectivity to CH₄ individually is less than about 5%, preferably less than about 0.5%, in certain embodiments.

According to some embodiments, the catalyst can comprise at least one Group IB element, at least one Group IIB element, and at least one Group IIIA element. For example, the Group IB element can be Cu, the Group IIB element can be Zn, and the Group IIIA element can be Al. The catalyst can further comprise at least one Group IA element, such as K or Cs. One catalyst that can be employed is Cu—Zn—Al—Cs.

In various embodiments, methods of the invention can use a H₂/CO ratio (from (ii) above) from about 0.5-4.0, preferably about 1.0-3.0, more preferably about 1.5-2.5. The average reactor temperature can be from about 200-400° C., preferably about 250-350° C. The average reactor pressure can be from about 20-500 atm, preferably about 50-200 atm. The average reactor residence time can be from about 0.1-10 seconds, preferably about 0.5-2 seconds.

Methanol produced, and/or syngas unreacted or produced from methanol, can be recycled back to the reactor. The methods can include at least two, three, or more recycle passes, which can be effective to increase at least one C₂-C₄ alcohol product selectivity to at least 50%, preferably at least 65%, and most preferably at least 80%.

In some embodiments of the present invention, the C₂-C₄ alcohols produced include ethanol, which can be (but not necessarily is) the most-selective reaction product.

In another aspect of the present invention, methods are provided for producing at least one C₂-C₄ alcohol from syngas, the method comprising:

(i) providing a reactor comprising a catalyst capable of converting syngas to alcohols;

(ii) providing a first stream containing a first amount of syngas;

(iii) flowing the first stream into the reactor at reaction conditions effective for producing a second stream comprising methanol and the at least one C₂-C₄ alcohol from the first amount of syngas, wherein the combined reaction selectivity to CO₂ and CH₄ is less than about 10%;

(iv) separating at least some methanol from the second stream;

(v) recycling at least some of the methanol back to the reactor;

(vi) reaching at least 90% of the equilibrium conversion from methanol to syngas in at least a portion of the reactor, wherein under the reactor conditions the equilibrium favors syngas, thereby generating a second amount of syngas from the methanol;

(vii) producing the at least one C₂-C₄ alcohol from the second amount of syngas; and

(viii) collecting a mixture comprising the at least one C₂-C₄ alcohol, wherein the mixture includes the alcohol produced in both steps (iii) and (vii).

In some embodiments, step (vi) reaches at least 95% of the equilibrium conversion. The conversion can reach equilibrium, or a conversion that is very close to the equilibrium-predicted value.

The methods can further comprise separating at least some unreacted syngas from the second stream, and recycling at least some of the unreacted syngas back to the reactor.

In some embodiments, the C₂-C₄ alcohols collected in step (viii) include an ethanol product selectivity of at least 50%, preferably at least 65%, and most preferably at least 80%.

In another aspect of the present invention, methods are provided for producing at least one C₂-C₄ alcohol from syngas, the method comprising:

(i) providing a reactor comprising a catalyst capable of converting syngas to alcohols;

(ii) providing a first stream containing a first amount of syngas;

(iii) flowing the first stream into the reactor at reaction conditions effective for producing a second stream comprising methanol and at least one C₂-C₄ alcohol from the first amount of syngas, in an amount described by reaction selectivity, wherein the combined reaction selectivity to CO₂ and CH₄ is less than about 10%;

(iv) separating at least some methanol from the second stream;

(v) recycling at least some of the methanol back to the reactor, wherein some of the methanol converts to a second amount of syngas;

(vii) producing the at least one C₂-C₄ alcohol from the second amount of syngas; and

(viii) collecting a product mixture comprising the at least one C₂-C₄ alcohol, in an amount described by product selectivity,

wherein the ratio of product selectivity to reaction selectivity for the at least one C₂-C₄ alcohol is about 1.25 or greater.

The ratio of product selectivity to reaction selectivity for the at least one C₂-C₄ alcohol can be at least about 1.5, 2, or greater. Of the at least one C₂-C₄ alcohol, ethanol can be most abundant.

In any of these method aspects of the invention, the combined reaction selectivity to CO₂ and CH₄ is preferably less than about 5%, such as 4%, 3%, 2%, 1%, or less than about 1%. The reaction selectivity to CO₂ itself is preferably less than about 5%, 4%, 3%, 2%, 1%, 0.5%, or even less, including essentially no CO₂ production. The reaction selectivity to CH₄ itself is preferably less than about 5%, 4%, 3%, 2%, 1%, 0.5%, or even less, including essentially no CH₄ production.

In preferred methods of the invention, at least one C₂-C₄ alcohol is produced in a product yield of at least 30%, preferably at least 40%, and more preferably at least 50%. It is generally desired to maximize the amount of carbon going to a single product, such as ethanol. However, in some embodiments, more than one C₂-C₄ alcohol is desired. In this case, the combined yield of desired products is preferably at least 30%, more preferably at least 40%, and most preferably at least 50%, along with the desired minimization of CO₂ and CH₄ as recited in the preceding paragraph.

A particular embodiment of the present invention provides a method for producing ethanol from syngas, the method comprising:

(i) providing a reactor comprising a catalyst containing copper, zinc, aluminum, and optionally cesium or potassium;

(ii) providing a first stream containing syngas having a H₂/CO ratio of 0.5-1.5;

(iii) flowing the first stream into the reactor at reaction conditions effective for producing a second stream comprising methanol and ethanol, wherein the combined reaction selectivity to CO₂ and CH₄ is less than about 1%;

(iv) separating at least some unreacted syngas from the second stream;

(v) separating at least some methanol from the second stream;

(vi) recycling at least some of the unreacted syngas and some of the methanol back to the reactor; and

(vii) reaching at least 90% of the equilibrium conversion from methanol to syngas in at least a portion of the reactor, wherein under the reactor conditions the equilibrium favors syngas, thereby generating a second amount of syngas from the methanol;

(viii) producing some ethanol from the second amount of syngas;

(ix) collecting a mixture that includes at least some ethanol produced in both steps (iii) and (viii); and

(x) collecting a product mixture comprising ethanol with product selectivity of at least 50%.

Another aspect of the invention provides an apparatus capable of carrying out any of the aforementioned methods. For example, in some embodiments, the apparatus is capable of producing at least one C₂-C₄ alcohol (such as ethanol) from syngas, the apparatus comprising:

(i) means for providing a first stream containing syngas;

(ii) a reactor comprising a catalyst, wherein:
(a) the catalyst is capable of converting syngas in the first stream into C₂-C₄ alcohols in a second stream;
(b) the catalyst is capable, at the same conditions in (ii)(a), of producing a reaction selectivity to CO₂ plus CH₄ of less than about 10% in the second stream;

(iii) means for separating at least some unreacted syngas from the second stream, and recycling the syngas back to the reactor;

(iv) means for separating at least some methanol from the second stream, and recycling the methanol back to the reactor; and

(v) means for purifying at least one C₂-C₄ alcohol produced in the reactor.

The catalyst employed in this apparatus can include at least one Group IB element such as Cu, at least one Group IIB element such as Zn, and at least one Group IIIA element such as Al. The catalyst can further include at least one Group IA element such as K or Cs.

### BRIEF DESCRIPTION OF THE FIGURES

FIG. 1 is a simplified process-flow diagram depicting one illustrative embodiment of the present invention.

FIG. 2 is a simplified process-flow diagram depicting another illustrative embodiment of the present invention.

FIG. 3 is a simplified process-flow diagram depicting another illustrative embodiment of the present invention.

### DETAILED DESCRIPTION OF EMBODIMENTS OF THE INVENTION

This description will enable one skilled in the art to make and use the invention. Several embodiments, adaptations, variations, alternatives, and uses of the invention, including what is presently believed to be the best mode of carrying out the invention, are described herein. As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon the specific analytical technique. Any numerical value inherently contains certain errors necessarily resulting from the standard deviation found in its respective testing measurements.

As used herein, “C₂-C₄ alcohols” means one or more alcohols selected from ethanol, propanol, and butanol, including all known isomers of such compounds. While preferred embodiments are described in relation to high selectivities to ethanol, the invention can also be practiced in a manner that gives high selectivities to propanol and/or butanol, or certain combinations of selectivities to ethanol, propanol, and butanol, depending on the desired fuel attributes. Methanol, according to preferred embodiments of the present invention, is not a desired product but rather an intermediate that can undergo further reactions to produce C₂-C₄ alcohols. It should be noted, however, that even when methanol is primarily used as a reactive intermediate, it can also be captured and sold in various quantities.

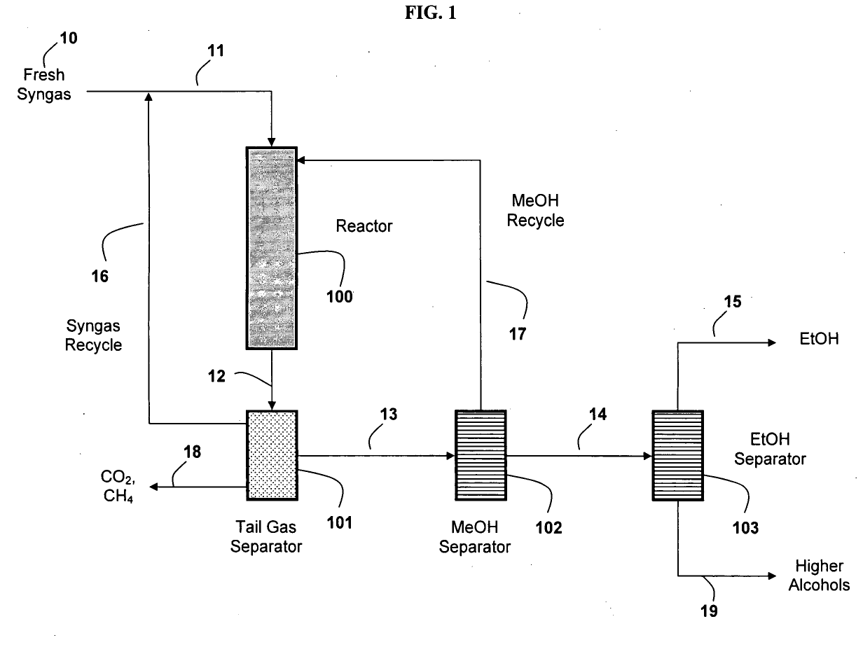

The present invention will now be described by reference to the following detailed description and accompanying drawings (FIGS. 1-3), which characterize and illustrate some preferred embodiments for producing ethanol. This description by no means limits the scope and spirit of the present invention. In the drawings, identical reference numbers refer to like elements. Two-digit numbers identify process streams, while three-digit numbers identify an apparatus, or means, for carrying out a chemical operation on the process stream(s).

With reference to the simplified process-flow diagram shown in FIG. 1, a stream 10 comprising syngas is fed to a reactor 100. The syngas stream 10 can be fresh syngas from a reformer or other apparatus, or can be recovered, recycled, and/or stored syngas. Stream 11 includes recycled syngas 16 (described below) and feeds the reactor 100. In some embodiments, the fresh syngas 10 is produced according to methods described in Klepper et al., “METHODS AND APPARATUS FOR PRODUCING SYNGAS,” U.S. patent application Ser. No. 12/166,167 (filed Jul. 1, 2008), the assignee of which is the same as the assignee of the present application. U.S. patent application Ser. No. 12/166,167 is hereby incorporated by reference herein in its entirety.

In some variations, stream 10 is filtered, purified, or otherwise conditioned prior to being introduced into reactor 100. For example, organic compounds, sulfur compounds, carbon dioxide, metals, and/or other impurities or potential catalyst poisons may be removed from syngas feed 10 (or may have been previously removed so as to produce stream 10) by conventional methods known to one of ordinary skill in the art. In some embodiments, any reaction byproducts can be returned to a reformer or other apparatus for producing additional syngas that can re-enter the process within stream 10.

The reactor 100 is any apparatus capable of being effective for producing at least one C₂-C₄ alcohol from the syngas stream feed. The reactor can be a single vessel or a plurality of vessels. The reactor contains at least one catalyst composition that tends to catalyze the conversion of syngas into C₂ and higher alcohols. For example, the reactor can contain a composition comprising Cu—Zn—Al—Cs, or another catalyst as described below.

Process stream 12 exits the reactor 100 and enters a tail gas separator 101. The tail gas separator 101 comprises a means for conducting a liquid-vapor separation at conditions similar to the conditions of reactor 100 or at some other conditions. The tail gas separator 101 further comprises a means for separating syngas from CO₂ and CH₄, to at least some extent, so that CO₂ and CH₄ (if produced) can be purged from tail gas separator 101 as shown in FIG. 1.

“Separator 101” can be a single separation device or a plurality of devices. For example, separator 101 can be a simple catchpot in which non-condensable gases are disengaged. Separator 101 can be a flash tank, multistage flash vessel, or distillation column, or several of such units, wherein the temperature and/or pressure are adjusted to different values after the reactor. Separator 101 can use a basis for separation other than relative volatilities, such as diffusion through pores or across membranes; solubility-diffusion across a solid phase; solubility-diffusion through a second liquid phase other than the liquid phase containing the C₂-C₄ alcohols; centrifugal force; and other means for separation as known to a skilled artisan.

When it is desired to remove CO₂ within separator 101, an absorption column can be used with, for example, an amine solvent. Alternately, pressure-swing adsorption can be used. Either of these options can remove at least some CO₂ and/or CH₄ from stream 12 and reject the CO₂ and/or CH₄ to stream 18. In certain embodiments wherein low amounts of CO₂ and CH₄ are produced by the catalyst, stream 18 may be small, including zero flow rate (i.e., all vapors from separator 101 can be recycled to reactor 100).

Stream 16 exiting the tail gas separator 101 comprises syngas that is not converted inside the reactor 100 in the instant reactor pass. The unconverted syngas 16 is recycled back to a point upstream of the reactor and combined with fresh feed 10 to produce mixed stream 11 which comprises fresh plus recycled syngas, and any impurities. The amount of syngas recycled in 16, and the recycle ratio of syngas (flow rate of stream 16 divided by flow rate of stream 11), will depend on the per-pass conversion realized in reactor 100 and the efficiency of separation in separator 101. The recycle ratio can be between 0 (no recycle) and 1 (no fresh feed). In various embodiments, the recycle ratio of syngas is at least about 0.1, 0.2, 0.3, 0.4, 0.5, 0.6, 0.7, 0.8, 0.9, or higher.

Stream 13 containing at least one alcohol exits the tail gas separator 101 and enters the methanol separator 102. In separator 102, a methanol recycle stream 17 that is enriched in methanol is removed. Stream 17 is recycled back to reactor 100, near the feed location according to the one embodiment shown in FIG. 1. Methanol separator 102 can be a flash tank or column or a distillation column, or multiple columns, as is known in the art. Methanol separation can generally be achieved by exploiting differences in volatility between methanol and other components present, or by using adsorption-based separation processes. Adsorption-based separation can use media including mesoporous solids, activated carbons, zeolites, and other materials known in the art.

The other stream 14 produced by unit 102 will generally contain most of the ethanol that was produced in reactor 100. In this example, stream 14 is sent forward to the ethanol separator 103. One of ordinary skill in the art will recognize that there are a variety of means for conducting the separation in ethanol separator 103. A flash tank or column can be used. When a plurality of separation stages are desired, distillation can be effective. Ethanol separation can be achieved by exploiting differences in volatility between ethanol and other components present, or by using adsorption-based separation processes, similar to methanol removal described above. Ethanol (contained in stream 15) is the primary product in this embodiment. Separator 103 also produces stream 19 comprising C₃₊ alcohols and possibly other oxygenates such as aldehydes, ketones, organic acids, and so on.

The recycled methanol 17 enters the reactor 100 preferably (but not necessarily) near the entrance. The methanol 17 and syngas 11 are expected to mix near the reactor entrance and will be subject to the well-known equilibrium between methanol and syngas (CO+2 H₂ [symbol] CH₃OH). For this equilibrium in the direction of methanol formation, the free energy of reaction is negative and the equilibrium constant is therefore higher (favoring methanol) at lower temperatures. Due to the mole-number change in the reaction, as pressure increases, equilibrium methanol formation will increase in accordance with Le Chatelier's principle.

As syngas concentration (partial pressure) increases, methanol formation increases. Alternately, as methanol concentration increases, the reaction is shifted to the left, towards syngas. When significant quantities of methanol are recycled, some portion of the recycled methanol can convert to CO and H₂. The distribution between methanol and syngas will depend on the reactor temperature and pressure; the inlet concentrations of methanol, syngas, and other species; and the extent of approach to equilibrium.

Relatively high levels of methanol near the reactor entrance can help prevent further production of methanol from syngas, thereby channeling syngas to ethanol and other C₂₊ products. Also, if the methanol-syngas reaction is at or near equilibrium, then (i) as syngas is consumed to produce ethanol and higher alcohols, and/or (ii) as additional methanol is introduced, Le Chatelier's principle would predict additional production of syngas from methanol.

Methanol can essentially serve as a liquid form of syngas whose hydrogen-carbon monoxide ratio is H₂/CO=2. When methanol is recycled, or when additional methanol is otherwise introduced, it can function similarly to recycled syngas. Stated differently, production of methanol by the catalyst does not necessarily reduce the ultimate selectivity or yield to ethanol or another desired C₂₊ alcohol, when methanol can be separated efficiently.

In the methods of the invention, the reactor 100 is operated at conditions effective for producing alcohols from syngas. In the apparatus of the invention, the reactor 100 is capable of being operated at conditions effective for producing alcohols from syngas. The phrase “conditions effective for producing alcohols from syngas” will now be described in detail.

Any suitable catalyst or combination of catalysts may be used in reactor 100 to catalyze reactions converting syngas to alcohols. Suitable catalysts for use in reactor 100 may include, but are not limited to, those disclosed in co-pending and commonly assigned U.S. Patent App. No. 60/948,653. Preferred catalysts minimize the formation of CO₂ and CH₄ under reaction conditions. In some embodiments, effective catalyst compositions comprise at least one Group IB element, at least one Group IIB element, and at least one Group IIIA element. Group IB elements are Cu, Ag, and Au. Group IIB elements are Zn, Cd, and Hg. Group IIIA elements are B, Al, Ga, In, and Tl. In certain embodiments, catalyst compositions further include at least one Group IA element. Group IA includes Li, Na, K, Rb, Cs, and Fr.

In a specific embodiment, the catalyst is a copper-zinc-aluminum-cesium (Cu—Zn—Al—Cs) catalyst. Such a catalyst composition can be prepared by adding cesium, using for example incipient wetness, to a commercial methanol-synthesis catalyst. Examples of commercial methanol-synthesis catalysts are those in the Katalco 51-series (51-8, 51-8PPT, and 51-9) available from Johnson Matthey Catalysts (U.S.A.).

In some embodiments, conditions effective for producing alcohols from syngas include a feed hydrogen-carbon monoxide molar ratio (H₂/CO) from about 0.2-4.0, preferably about 0.5-2.0, and more preferably about 0.5-1.5. These ratios are indicative of certain embodiments and are not limiting. It is possible to operate at feed H₂/CO ratios less than 0.2 as well as greater than 4, including 5, 10, or even higher. It is well-known that high H₂/CO ratios can be obtained with extensive steam reforming and/or water-gas shift in operations prior to the syngas-to-alcohol reactor.

In embodiments wherein H₂/CO ratios close to 1:1 are desired for alcohol synthesis, partial oxidation of the carbonaceous feedstock can be utilized, at least in part, to produce stream 10. In the absence of other reactions, partial oxidation tends to produce H₂/CO ratios close to unity, depending on the stoichiometry of the feedstock.

When, as in certain embodiments, relatively low H₂/CO ratios are desired, the reverse water-gas shift reaction (H₂+CO₂→H₂O+CO) can potentially be utilized to consume hydrogen and thus lower H₂/CO. In some embodiments, CO₂ produced during alcohol synthesis, or elsewhere, can be recycled to the reformer to decrease the H₂/CO ratio entering the alcohol-synthesis reactor. Other chemistry and separation approaches can be taken to adjust the H₂/CO ratios prior to converting syngas to alcohols, as will be appreciated.

In some embodiments, feed H₂/CO refers to the composition of stream 10, which is the feed to the process of the invention. In other embodiments, feed H₂/CO refers to the composition of stream 11 (with syngas recycle), which is the reactor feed. In still other embodiments, feed H₂/CO refers to the composition of the reactor contents after recycled methanol is injected and after methanol-syngas equilibrium is substantially reached, and before the resulting mixture “feeds” a kinetically controlled region of the catalyst. In the latter case, it is noted that methanol stoichiometrically converts to H₂/CO=2 and can therefore adjust the actual ratio upward or downward, depending on what the H₂/CO ratio is prior to methanol injection.

In some embodiments, conditions effective for producing alcohols from syngas include reactor temperatures from about 200-400° C., preferably about 250-350° C. Certain embodiments employ reactor temperatures of about 280° C., 290° C., 300° C., 310° C., or 320° C. Depending on the catalyst chosen, changes to reactor temperature can change conversions, selectivities, and catalyst stability. As is recognized in the art, increasing temperatures can sometimes be used to compensate for reduced catalyst activity over long operating times.

Preferably, the syngas entering the reactor is compressed. Conditions effective for producing alcohols from syngas include reactor pressures from about 20-500 atm, preferably about 50-200 atm or higher. Generally, productivity increases with increasing reactor pressure, and pressures outside of these ranges can be employed with varying effectiveness.

In some embodiments, conditions effective for producing alcohols from syngas include average reactor residence times from about 0.1-10 seconds, preferably about 0.5-2 seconds. “Average reactor residence time” is the mean of the residence-time distribution of the reactor contents under actual operating conditions. Catalyst contact times can also be calculated by a skilled artisan and these times will typically also be in the range of 0.1-10 seconds, although it will be appreciated that it is certainly possible to operate at shorter or longer times.

The reactor for converting syngas into alcohols can be engineered and operated in a wide variety of ways. The reactor operation can be continuous, semicontinuous, or batch. Operation that is substantially continuous and at steady state is preferable. The flow pattern can be substantially plug flow, substantially well-mixed, or a flow pattern between these extremes. The flow direction can be vertical-upflow, vertical-downflow, or horizontal. A vertical configuration can be preferable.

The “reactor” can actually be a series or network of several reactors in various arrangements. For example, in some variations, the reactor comprises a large number of tubes filled with one or more catalysts.

The catalyst phase can be a packed bed or a fluidized bed. The catalyst particles can be sized and configured such that the chemistry is, in some embodiments, mass-transfer-limited or kinetically limited. The catalyst can take the form of powder, pellets, granules, beads, extrudates, and so on. When a catalyst support is optionally employed, the support may assume any physical form such as pellets, spheres, monolithic channels, etc. The supports may be coprecipitated with active metal species; or the support may be treated with the catalytic metal species and then used as is or formed into the aforementioned shapes; or the support may be formed into the aforementioned shapes and then treated with the catalytic species.

Reaction selectivities can be calculated on a carbon-atom basis. “Carbon-atom selectivity” means the ratio of the moles of a specific product to the total moles of all products, scaled by the number of carbon atoms in the species. This definition accounts for the mole-number change due to reaction, and best describes the fate of the carbon from converted CO. The selectivity Sⱼ to general product species Cₓ_{ⱼ}H_{y}_{ⱼ}O_{z}_{ⱼ} is

$${S}_{j} = \frac{{x}_{j} {F}_{j}}{{\sum }_{i} {x}_{i} {F}_{i}}$$

wherein Fⱼ is the molar flow rate of species j which contains xⱼ carbon atoms. The summation is over all carbon-containing species (Cₓ_{ᵢ}H_{y}_{ᵢ}O_{z}_{ᵢ}) produced in the reaction. In some embodiments, wherein all products are identified and measured, the individual selectivities sum to unity (plus or minus analytical error). In other embodiments, wherein one or more products are not identified in the exit stream, the selectivities can be calculated based on what products are in fact identified, or instead based on the conversion of CO.

For the purpose of clarifying the present invention, “reaction selectivity” describes the per-pass selectivity governing the catalysis from syngas to products. “Product selectivity” is the net selectivity for the process—what is observed in the total process output (e.g., streams 15, 18, and 19 shown in FIG. 1). Product selectivity, as intended herein, is a hybrid parameter that accounts for not only catalyst performance but also process integration and recycle efficiency.

As a hypothetical example for illustration purposes only, a process according to FIG. 1 producing 6 moles ethanol and 1 mole methanol in stream 15, 1 mole propanol and 1 mole butanol in stream 19, and 1 mole CO₂ in stream 18 would have an ethanol product selectivity of 2×6/(2×6+1×1+3×1+4×1+1×1)=57.1%. Other product selectivities for this calculation example are as follows: methanol=4.8%; propanol=14.3%; butanol=19.0%; and CO₂=4.8%.

In various embodiments of the present invention, the product stream from the reactor may be characterized by reaction selectivities of about 10-60% or higher to methanol and about 10-50% or higher to ethanol. The product stream from the reactor may include up to, for example, about 25% reaction selectivity to C₃₊ alcohols, and up to about 10% to other non-alcohol oxygenates such as aldehydes, esters, carboxylic acids, and ketones. These other oxygenates can include, for example, acetone, 2-butanone, methyl acetate, ethyl acetate, methyl formate, ethyl formate, acetic acid, propanoic acid, and butyric acid.

According to the present invention, when methanol recycle is taken into account, the net selectivity to ethanol can be higher (preferably substantially higher) than the net selectivity to methanol. In preferred embodiments, the ethanol product selectivity is higher, preferably substantially higher, than the methanol product selectivity, such as a product selectivity ratio of ethanol/methanol of about 1, 2, 3, 4, 5 or higher. The product selectivity ratio of ethanol to all other alcohols is preferably at least 1, more preferably at least 2, 3, 4 or higher.

As methanol is recycled, the ethanol product selectivity according to embodiments of the invention can reach at least about 50%, 55%, 60%, 65%, 70%, 75%, 80% or even higher, when the selected catalyst produces low amounts of carbon dioxide, methane, and higher alcohols and other oxygenates. In the methods of the invention, the yield of ethanol can be defined as the moles of carbon in ethanol divided by moles of carbon in fresh-feed CO. With ideal methanol separation and sufficient recycle, the ethanol yields can in principle approach the ethanol product selectivities as recited in the paragraph above.

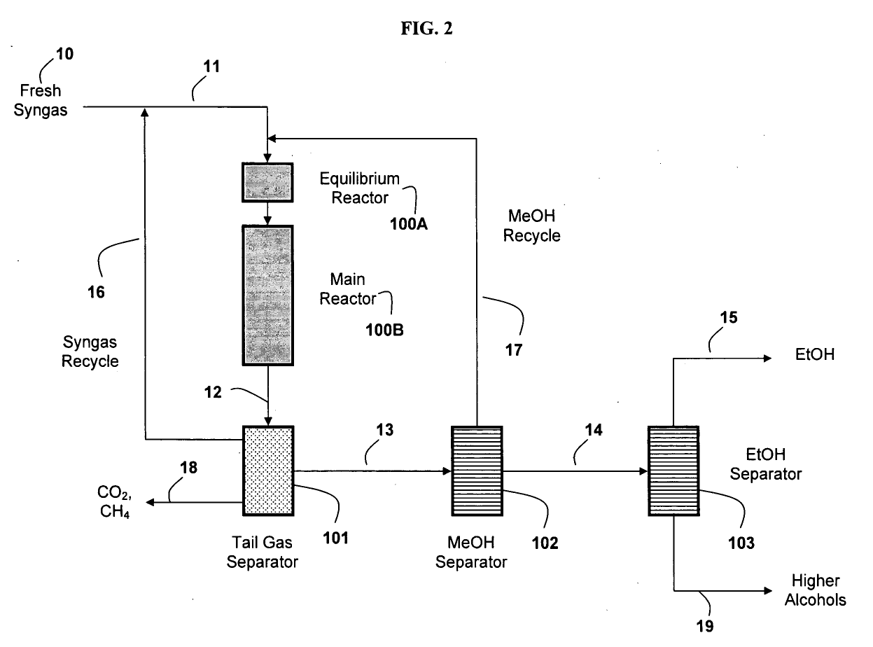

Other embodiments of the present invention can be understood by reference to FIG. 2. The primary difference with the embodiments depicted in FIG. 1 is that the reactor consists of an equilibrium reactor 100A and a primary reactor 100B that are physically separated. In 100A, the recycled methanol is allowed to come to its equilibrium distribution with CO and H₂, which in preferred embodiments is net generation of syngas from methanol. This equilibrium mixture is then fed to the main unit 100B. One advantage of this aspect is that by splitting the reactors 100A and 100B, different process conditions can be used. For example, 100A could be operated at relatively low pressure or high temperature to favor syngas formation from methanol. Generally speaking, conditions in both reactors 100A and 100B can be independently selected according to the description of reactor 100 conditions above.

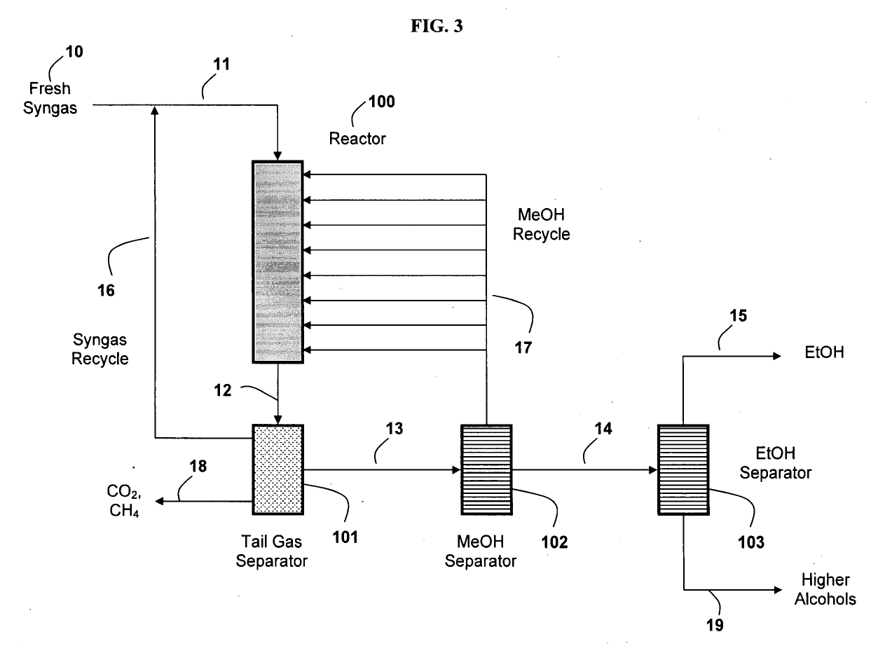

Still other embodiments of the present invention can be understood by reference to FIG. 3. These embodiments are premised on the realization that it can be advantageous to inject recycled methanol not just at the reactor 100 inlet, but throughout the reaction zone. In this way, methanol formation from syngas can be suppressed, thereby channeling syngas to ethanol and higher alcohols, along the entire length of the catalyst bed. Effective operating conditions for reactor 100 in FIG. 3 are expected to be reasonably similar to those described above with respect to FIG. 1.

In general, the specific selection of catalyst configuration (geometry), H₂/CO ratio, temperature, pressure, and residence time (or feed rate) will be selected to provide, or will be subject to constraints relating to, an economically optimized process. The plurality of reactor variables and other system parameters can be optimized, in whole or in part, by a variety of means. For example, statistical design of experiments can be carried out to efficiently study several variables, or factors, at a time. From these experiments, models can be constructed and used to help understand certain preferred embodiments. An illustrative statistical model that might be developed is ethanol selectivity vs. several factors and their interactions. Another model might relate to combined CO₂+CH₄ selectivity, a parameter that is preferably minimized herein.

In some embodiments, it can be desirable to first select a catalyst system and then to proceed with optimizing reactor operation with the initial catalyst composition as a fixed parameter. It is well within the capability of a person of ordinary skill in the arts of catalysis and reactor engineering to optimize the systems of the invention in this manner.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A method for producing at least one C₂-C₄ alcohol from syngas, the method comprising:
(i) providing a reactor comprising a catalyst capable of converting syngas to alcohols;
(ii) providing a first stream containing syngas having a H₂/CO ratio;
(iii) flowing said first stream into said reactor at reaction conditions effective for producing a second stream comprising methanol and said at least one C₂-C₄ alcohol, wherein the combined reaction selectivity to CO₂ and CH₄ is less than about 10%;
(iv) separating at least some unreacted syngas from said second stream;
(v) separating at least some methanol from said second stream;
(vi) recycling at least some of said unreacted syngas and some of said methanol back to said reactor; and
(vii) collecting a mixture comprising said at least one C₂-C₄ alcohol.

2. The method of claim 1, wherein in step (iii) said combined reaction selectivity to CO₂ and CH₄ is less than about 5%.

3. The method of claim 2, wherein said combined reaction selectivity to CO₂ and CH₄ is less than about 1%.

4. The method of claim 1, wherein in step (iii) the reaction selectivity to CO₂ is less than about 5%.

5. The method of claim 4, wherein said reaction selectivity to CO₂ is less than about 0.5%.

6. The method of claim 5, wherein said reaction selectivity to CO₂ is essentially 0.

7. The method of claim 1, wherein in step (iii) the reaction selectivity to CH₄ is less than about 5%.

8. The method of claim 7, wherein said reaction selectivity to CH₄ is less than about 0.5%.

9. The method of claim 1, wherein said at least one C₂-C₄ alcohol includes ethanol.

10. The method of claim 9, wherein said ethanol is the most-selective reaction product.

11. The method of claim 1, wherein said catalyst comprises at least one Group IB element, at least one Group IIB element, and at least one Group IIIA element.

12. The method of claim 11, wherein at least one Group IB element is Cu, at least one Group IIB element is Zn, and at least one Group IIIA element is Al.

13. The method of claim 11, wherein said catalyst further comprises at least one Group IA element.

14. The method of claim 13, wherein at least one Group IB element is Cu, at least one Group IIB element is Zn, at least one Group IIIA element is Al, and at least one Group IA element is either K or Cs.

15. The method of claim 1, wherein the catalyst is Cu—Zn—Al—Cs.

16. The method of claim 1, wherein said H₂/CO ratio is from about 0.5 to about 4.0.

17. The method of claim 16, wherein said H₂/CO ratio is from about 1.0 to about 3.0.

18. The method of claim 17, wherein said H₂/CO ratio is from about 1.5 to about 2.5.

19. The method of claim 1, wherein the average reactor temperature is from about 200° C. to about 400° C.

20. The method of claim 19, wherein the average reactor temperature is from about 250° C. to about 350° C.

21. The method of claim 1, wherein the average reactor pressure is from about 20 atm to about 500 atm.

22. The method of claim 21, wherein said average reactor pressure is from about 50 atm to about 200 atm.

23. The method of claim 1, wherein the average reactor residence time is from about 0.1 seconds to about 10 seconds.

24. The method of claim 23, wherein said average reactor residence time is from about 0.5 seconds to about 2 seconds.

25. The method of claim 1, said method comprising at least two recycle passes.

26. The method of claim 25, said method comprising at least three recycle passes.

27. The method of claim 1, said method comprising a plurality of recycle passes effective to increase at least one C₂-C₄ alcohol product selectivity to at least 50%.

28. The method of claim 27, wherein said product selectivity is increased to at least 65%.

29. The method of claim 28, wherein said product selectivity is increased to at least 80%.

30. A method for producing at least one C₂-C₄ alcohol from syngas, the method comprising:
(i) providing a reactor comprising a catalyst capable of converting syngas to alcohols;
(ii) providing a first stream containing a first amount of syngas;
(iii) flowing said first stream into said reactor at reaction conditions effective for producing a second stream comprising methanol and said at least one C₂-C₄ alcohol from said first amount of syngas, wherein the combined reaction selectivity to CO₂ and CH₄ is less than about 10%;
(iv) separating at least some methanol from said second stream;
(v) recycling at least some of said methanol back to said reactor;
(vi) reaching at least 90% of the equilibrium conversion from methanol to syngas in at least a portion of said reactor, wherein under the reactor conditions said equilibrium favors syngas, thereby generating a second amount of syngas from said methanol;
(vii) producing said at least one C₂-C₄ alcohol from said second amount of syngas; and
(viii) collecting a mixture comprising said at least one C₂-C₄ alcohol, wherein said mixture includes said alcohol produced in both steps (iii) and (vii).

31. The method of claim 30, wherein step (vi) reaches at least 95% of said equilibrium conversion.

32. The method of claim 30, wherein step (vi) substantially reaches a conversion predicted by equilibrium.

33. The method of claim 30, further comprising separating at least some unreacted syngas from said second stream, and recycling at least some of said unreacted syngas back to said reactor.

34. The method of claim 30, wherein the C₂-C₄ alcohols collected in step (viii) include an ethanol product selectivity of at least 50%.

35. The method of claim 34, wherein said ethanol product selectivity is at least 65%.

36. The method of claim 35, wherein said ethanol product selectivity is at least 80%.

37. A method for producing at least one C₂-C₄ alcohol from syngas, the method comprising:
(i) providing a reactor comprising a catalyst capable of converting syngas to alcohols;
(ii) providing a first stream containing a first amount of syngas;
(iii) flowing said first stream into said reactor at reaction conditions effective for producing a second stream comprising methanol and at least one C₂-C₄ alcohol from said first amount of syngas, in an amount described by reaction selectivity, wherein the combined reaction selectivity to CO₂ and CH₄ is less than about 10%;
(iv) separating at least some methanol from said second stream;
(v) recycling at least some of said methanol back to said reactor, wherein some of said methanol converts to a second amount of syngas;
(vii) producing said at least one C₂-C₄ alcohol from said second amount of syngas; and
(viii) collecting a product mixture comprising said at least one C₂-C₄ alcohol, in an amount described by product selectivity,
wherein the ratio of product selectivity to reaction selectivity for said at least one C₂-C₄ alcohol is about 1.25 or greater.

38. The method of claim 37, wherein said ratio is about 1.5 or greater.

39. The method of claim 38, wherein said ratio is about 2 or greater.

40. The method of claim 37, wherein of said at least one C₂-C₄ alcohol, ethanol is most abundant.

41. The method of any of claims 1, 30, or 37, wherein in step (iii) the reaction selectivity to CO₂ is less than about 5% and the reaction selectivity to CH₄ is less than about 5%.

42. The method of any of claims 1, 30, or 37, wherein in step (iii) said combined reaction selectivity to CO₂ and CH₄ is less than about 5%.

43. The method of any of claims 1, 30, or 37, wherein at least one C₂-C₄ alcohol is produced in a product yield of at least 30%.

44. The method of claim 43, wherein said product yield is at least 40%.

45. The method of claim 44, wherein said product yield is at least 50%.

46. A method for producing ethanol from syngas, said method comprising:
(i) providing a reactor comprising a catalyst containing copper, zinc, aluminum, and optionally cesium or potassium;
(ii) providing a first stream containing syngas having a H₂/CO ratio selected from 0.5-1.5;
(iii) flowing said first stream into said reactor at reaction conditions effective for producing a second stream comprising methanol and ethanol, wherein the combined reaction selectivity to CO₂ and CH₄ is less than about 1%;
(iv) separating at least some unreacted syngas from said second stream;
(v) separating at least some methanol from said second stream;
(vi) recycling at least some of said unreacted syngas and some of said methanol back to said reactor; and
(vii) reaching at least 90% of the equilibrium conversion from methanol to syngas in at least a portion of said reactor, wherein under the reactor conditions said equilibrium favors syngas, thereby generating a second amount of syngas from said methanol;
(viii) producing some ethanol from said second amount of syngas;
(ix) collecting a mixture that includes at least some ethanol produced in both steps (iii) and (viii); and
(x) collecting a product mixture comprising ethanol with product selectivity of at least 50%.

47. An apparatus capable of producing at least one C₂-C₄ alcohol from syngas, said apparatus comprising:
(i) means for providing a first stream containing syngas;
(ii) a reactor comprising a catalyst, wherein (a) said catalyst is capable, under effective conditions, of converting syngas in said first stream into C₂-C₄ alcohols in a second stream, and (b) said catalyst is capable, at said effective conditions, of producing a reaction selectivity to CO₂ plus CH₄ of less than about 10% in said second stream;
(iii) means for separating at least some unreacted syngas from said second stream, and recycling said syngas back to said reactor;
(iv) means for separating at least some methanol from said second stream, and recycling said methanol back to said reactor; and
(v) means for purifying at least one C₂-C₄ alcohol produced in said reactor.

48. The apparatus of claim 47, wherein said at least one C₂-C₄ alcohol is ethanol.

49. The apparatus of claim 47, wherein said catalyst comprises at least one Group IB element, at least one Group IIB element, and at least one Group IIIA element.

50. The apparatus of claim 49, wherein at least one Group IB element is Cu, at least one Group IIB element is Zn, and at least one Group IIIA element is Al.

51. The apparatus of claim 49, wherein said catalyst further comprises at least one Group IA element.

52. The apparatus of claim 51, wherein at least one Group IB element is Cu, at least one Group IIB element is Zn, at least one Group IIIA element is Al, and at least one Group IA element is either K or Cs.
